(19) United States
(12) Patent Application Publication
(10) Pub. No.: US 2013/0323750 A1
(43) Pub. Date: Dec. 5, 2013

(54) Method for Analyzing Apoptosis Inducing Factor-2

(71) Applicant: National Taiwan University Hospital., Taipei City (TW)

(72) Inventors: Kuan-Tun Wu, Taipei City (TW); Ann Chen, Taipei City (TW); Shuk-Man Ka, Taipei City (TW)

(73) Assignee: National Taiwan University Hospital, Taipei City (TW)

(21) Appl. No.: 13/903,972

(22) Filed: May 28, 2013

(30) Foreign Application Priority Data
May 30, 2012 (TW) 101119376

(51) Int. Cl. G01N 33/68

(52) U.S. Cl. CPC G01N 33/6872
USPC 435/7.4

(57) ABSTRACT

A method for analyzing apoptosis inducing factor-2 (AIF-2) is disclosed, which has not been found in urine samples of human patients with chronic kidney disease, to establish AIF-2 as a non-invasive biomarker for chronic kidney disease. The method includes: collecting a plurality of urine samples; conducting western blot for each urine sample for detecting the AIF-2 protein content in each urine sample; and conducting statistical analysis of the AIF-2 protein content in each urine sample to establish AIF-2 as a biological marker for chronic kidney disease.

### BACKGROUND OF THE INVENTION

1. Field of the Invention

The present invention relates to an analyzing method of creating a biomarker, more particularly to a method for analyzing apoptosis inducing factor-2 (AIF-2), to create AIF-2 as a biomarker for chronic kidney disease. 2. Description of the Related Art

Nephritis, nephrotic syndrome, and renal degeneration disease are common diseases. The rapid increase in the number of patients who suffer from chronic kidney disease and need long-term dialysis worldwide has caused problems of social service, health, and medical resource allocation.

The above-mentioned diseases are listed as eighth on a list of the ten leading causes of death of people in Taiwan, and in the elderly, the increase is apparent. Due to the increase of kidney disease, the dialysis population in Taiwan has reached more than 45,000 people, which is a heavy burden for today's limited health care resources. Chronic kidney disease is classified according to the value of Glomerular Filtration Rate (GFR) into five phases, from Phase 1 (minor) to Phase 5 (most serious). The current prevention of chronic kidney disease focuses on early diagnosis and early treatment. However, when it is necessary that chronic kidney disease be diagnosed, invasive inspection of kidney tissue sections is usually required. Even though clinically, serum creatinine and urea nitrogen can be applied as a renal function index, they still cannot completely reflect the extent of kidney damage. As a result, an inspection method that has high judgment value, is non-invasive, and has high predictability is needed very urgently to help prevent chronic kidney disease in patients and provide appropriate medical care.

Due to the uniqueness and forward-looking perspectives of nephritis early biomarkers, in order to develop a non-invasive early diagnosis warning method, prior research studies have used the latest proteomics technologies to look for biomarkers possibly existing in blood, urine and tissues to achieve early diagnosis and early treatment.

Therefore, the successful development of innovative non-invasive biological indexes would be helpful in the early diagnosis and early treatment of chronic kidney diseases.

### SUMMARY OF THE INVENTION

The present invention discloses a method for analyzing apoptosis inducing factor-2 (AIF-2) for analyzing AIF-2 that has not been found in urine samples of human patients with chronic kidney disease to establish AIF-2 as a non-invasive biomarker for chronic kidney disease. The method includes: collecting a plurality of urine samples; conducting western blot for each urine sample for detecting the AIF-2 protein content in each urine sample; and conducting statistical analysis for the AIF-2 protein content in each urine sample to establish AIF-2 as a biological marker for chronic kidney disease.

### BRIEF DESCRIPTION OF THE DRAWINGS

FIG. 1 is the step flow chart of the method for analyzing apoptosis inducing factor-2 in this invention.

FIG. 2A shows the performance condition of the detected AIF2 protein of normal tissues in the immunohistochemical analysis staining according to the present invention.

FIG. 2B shows the performance condition of the detected AIF2 protein of chronic kidney disease Phase 3a tissues in the immunohistochemical analysis staining according to the present invention.

FIG. 3A shows the content of the urine samples of the detected AIF2 protein of normal tissues and chronic kidney disease Phase 1 to 5 in conducting the western blot transfer crushing analysis according to the present invention.

FIG. 3B shows the semi-quantitative analysis of the urine AIF-2 content of chronic kidney disease from Phase 1 to 5.

### DETAILED DESCRIPTION OF THE PREFERRED EMBODIMENT

To clarify the above and other purposes, features and advantages of this invention, the specific embodiment of this invention is especially listed and described in detail with the attached figures as follows.

In order to develop a non-invasive early diagnosis warning method, in this invention, urine and renal tissues of patients with chronic kidney disease are used and the latest proteomics technologies, including immunohistochemical staining analysis and western blot analysis, are applied for detecting apoptosis inducing factor-2 (AIF-2) in urine and tissues to make AIF-2 a biomarker for chronic kidney disease.

The apoptosis inducing factor-2 (AIF-2) in the present invention belongs to one of apoptosis-inducing factor protein families and is closely associated with apoptosis and inflammation.

The currently known functions of AIF-2 are:

1. Participating in non-dependent CASPASE apoptotic pathways: AIF-2 is an oxidoreductase, located in the mitochondrial ship. When cells receive death signals, AIF-2 will move from mitochondria to nucleus, where it makes chromosomes condense and causes DNA to be chipped into DNA fragments. AIF-2 is involved in oxidase activity of NAD(P)H and catalyzes the reduction of NAD (P) H-dependent Cytochrome C and other electronic receivers.

2. Tumor suppression: It has been found in human tumor tissues that the expression of the mRNA of AIF-2 is downward regulated; thus it is considered that AIF-2 has the potential to inhibit tumor growth. Yet AIF-2 has not been found in the urine of human patients with chronic kidney disease, so AIF-2 has non-invasive biomarker capabilities as an innovative development. In order to establish the apoptosis-inducing factor (AIF-2) as a biomarker for chronic kidney disease, the present invention discloses a method for analyzing apoptosis inducing factor-2 (AIF-2).

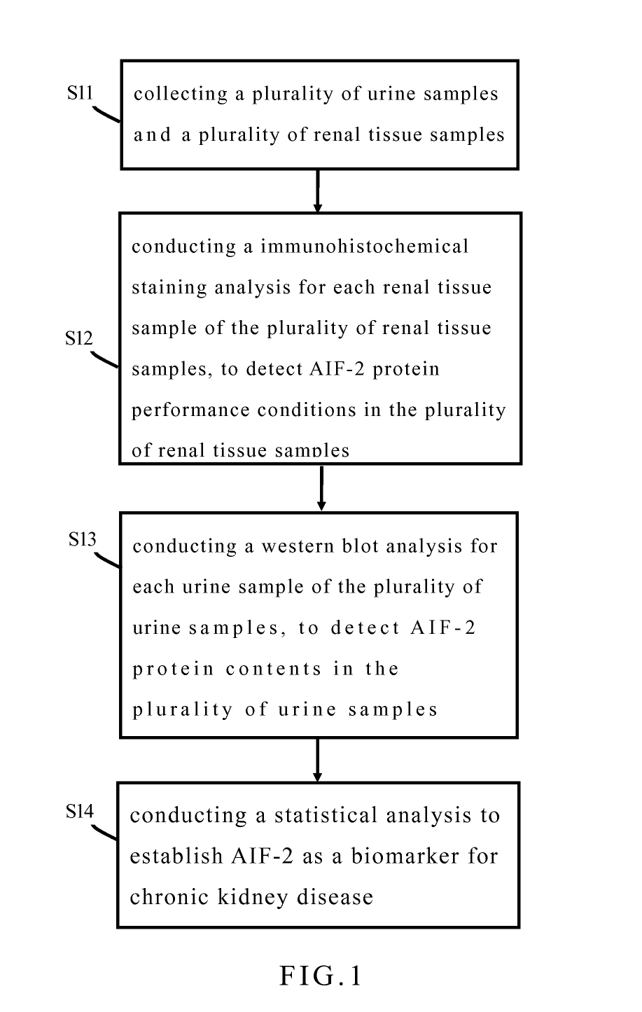

Hereinafter refer to FIG. 1, which is the step flow chart of the method for analyzing apoptosis inducing factor-2 in this invention.

First, in the present invention, step S11 is conducted to collect a plurality of renal tissue samples and urine samples. For example, as shown in the table below, in step S11 of the present invention, clinical laboratory data of 80 patients are collected. These patients belong to the first to the fifth level of chronic kidney disease (CKD) respectively, and their ages are distributed from 19 to 90. Their total cholesterol ranges from 109 to 729 mmol/L, Triglyceride 49˜620 mmol/L, Alanine aminotransferase (GPT) 8˜75 U/L, and Serum creatinine 0.6˜13.7 mg/dl.

| | CKDI | CKDII | CKDIII | CKDIV | CKDV |
|---|---|---|---|---|---|
| Age (year) | 25 | 50 | 65.5 | 53 | 64 |
| | (19-60) | (21-62) | (49-75) | (20-90) | (23~82) |
| Total | 167.5 | 225 | 206 | 186 | 182 |
| cholesterol | (109-729) | (126~608) | (119~403) | (134-440) | (125~288) |
| (mmol/L)) | | | | | |
| Tri- | 108.5 | 200 | 156 | 131 | 127 |
| glyceride | (49~432) | (83-620) | (49-405) | (68~215) | (89-223) |
| (mmol/L) | | | | | |
| GPT(U/L) | 15 | 17 | 16 | 21 | 15 |
| | (11-75) | (9~63) | (8-61) | (11-36) | (8-22) |
| Serum | 0.8 | 1.1 | 1.8 | 2.6 | 7.7 |
| creatinine | (0.6-1) | (0.8-1.3) | (1.1-2.2) | (2-3.4) | (3.6-13.7) |
| (mg/dl) | | | | | |

In one embodiment of the present invention, the urine and renal tissues of clinical patients with chronic kidney disease are obtained legally and under consent.

In the present invention, urine samples are placed on ice, then supernatants of the urine samples are obtained after centrifugation and the supernatant is respectively poured into a plurality of collection tubes. The renal tissue samples are placed in liquid nitrogen. Sources, names, ages, and diagnoses of the plurality of urine samples and the plurality of renal tissue samples are marked and all relevant clinical information is recorded. At the same time, in the present invention, clinical urinary protein analysis, renal function analysis, and pathological analysis are conducted on the samples. The records of the prior literature can be referenced to obtain knowledge of the determination of renal functions, histological examination, and the assessment and semi-quantitative analysis of immunohistochemical staining

Blood ureanitrogen (BUN), Creatinine (Cr), and proteinuria determinations are determined by an automatic analyzer. The change in hematuria is detected by Hema-Combistix strip. The kidney tissues are fixed by 10% formalin, sliced and stained with Hematoxylin and Eosin, and then the kidney tissues are observed under a microscope for detecting changes in kidney tissues. Then, in the present invention, the step S12 is conducted for the immunohistochemical staining analysis for each renal tissue sample of the plurality of renal tissue specimens for the detection of AIF-2 protein performance condition in the renal tissue samples.

In step S12, in the present invention, the tissues are placed in a 75° C. oven and baked for 30 minutes to melt the wax. The tissues are sliced and conducted with lost-wax in a automatic machine (LeicaAuto Stainer XL) and then washed by TBST (0.01% Tween 20) three times. The activity of

Endogenous Peroxidase is stopped by adding 3% H2O2 in Methanol to the tissue slice and reacting at room temperature for 10 minutes. Then the sample is cleaned by TBST twice, 2% BSA Blocking is added, and the sample is held at room temperature for 30 minutes. After that, the sample is cleaned by TBST 3 times, the tested primary antibody is added, and the sample is held at 4° C. for reaction until the following day. The next day, the sample is removed from the 4° C. refrigerator to warm for 1 hour. Then the sample is cleaned by TBST 3 times, Protein G-HRP (For LSAB system, HRP; DAKO) is added, and the sample is held at room temperature for 1 hour for reaction, after which it is washed 3 times by TBST (0.01% Tween 20). Then AEC+Substrate-Chromogen (DAKO) is added to the sample and the sample is held at room temperature for 10 minutes for reaction. The sample is cleaned by TBST 3 times and then soaked in the purple solution for 30 seconds for background staining Then the sample is cleaned with clean water and mounted after air drying to be observed under the microscope.

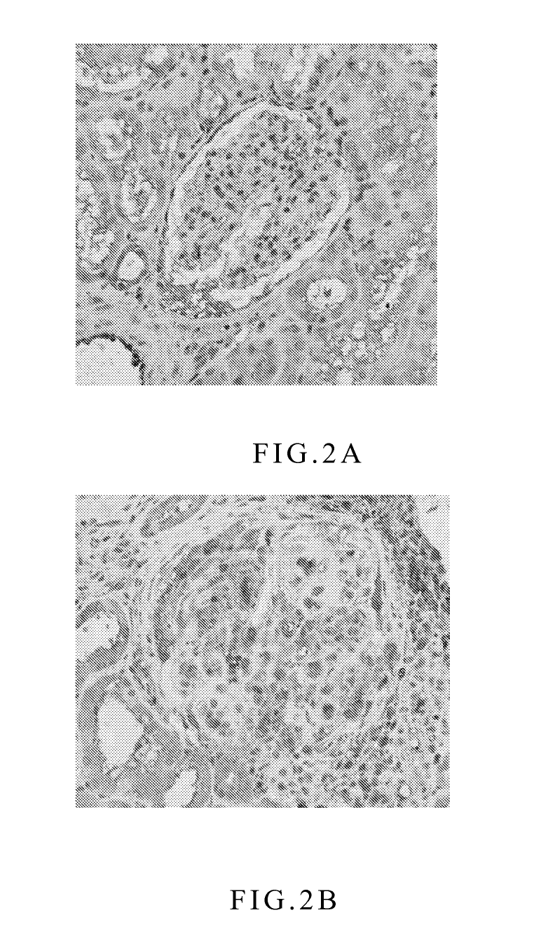

As shown in FIG. 2A and FIG. 2B, in accordance with the step S12, in which the immunohistochemical staining is conducted to detect AIF-2 protein changes, renal tissues of chronic kidney disease patients in different phases is analyzed, and the AIF-2 protein also has high performance in kidney cells of chronic kidney disease patients, which were significantly different from the renal tissues of normal patients. FIG. 2A shows normal tissues taken from the area surrounding kidney tumors. FIG. 2B shows the tissue sections of patients with chronic kidney disease phase IIIa (CKD IIIa).

Then, in the present invention, step S13 is conducted to analyze each urine sample with the western blot analysis method to detect the content of AIF-2 protein in the urine samples. In step S13 of the present invention, patient urine samples are centrifuged to obtain 35μl supernatant, and the supernatant is added with 6X sample buffer 7 μl, placed on ice for 10 minutes, and then injected into the well of the Stacking gel to conduct Protein Electrophoresis. The semi-dry transfer crushing machine (HorizBlot, AE6675, the ATTO, Japan) is used to separate the protein on the SDS-PAGE colloid to transfer crush it into the PVDF Sigma. The transferred PVDF is placed in 5% 1x TBST and skim milk for 2 hours, and then the first antibody is added in an appropriate concentration dilution and the sample is held at 4° C. until the next day for reaction. The next day, the 2000-times diluted secondary antibody combined with HRP is added and the mixture is shaken at room temperature for reaction for 60 minutes. The PVDF Sigma is added with ECL Plus (Amersham, UK), and light-sensitive film (Kodak BioMax Light, Film, Kodak,USA) is used for coloring.

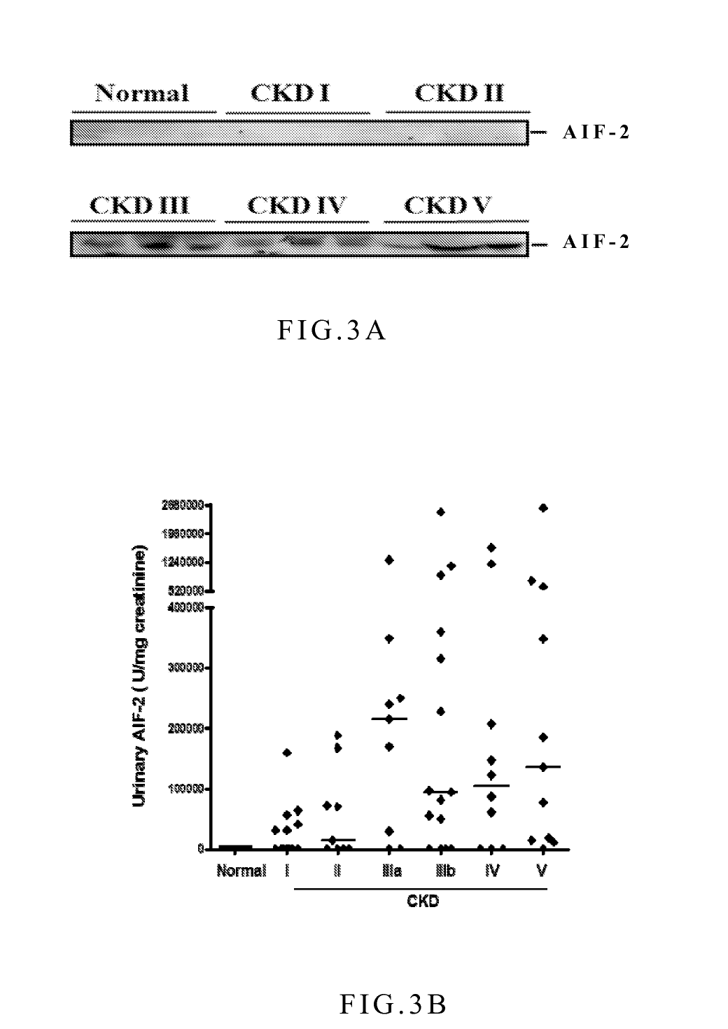

As shown in FIG. 3A and FIG. 3B, according to step S13, which analyzes whether the urine contains AIF-2 protein, the western blot transfer crushing method is conducted for chronic kidney disease patients in different phases, and it shows that the AIF-2 protein content in the urine of patients with chronic kidney disease is higher than that in normal patients, with significant differences. FIG. 3A respectively shows normal tissues taken from tissue surrounding a kidney tumor and tissue sections from patients with chronic kidney disease phase III (CKD III). FIG. 3B shows the semi-quantitative analysis of the urine AIF-2 content of chronic kidney disease from Phase 1 to 5.

Finally, step S14 is conducted in the present invention for statistical analysis of the performance condition of AIF-2 mRNA and protein in the plurality of renal tissue samples as well as the AIF-2 protein content in the plurality of urine samples, to create AIF-2 as a biomarker for chronic kidney disease. In step S14 of the present invention, statistical analysis is conducted for comparison between groups, as shown by the median and the nonparametric test and/or post-mortem analysis. The p value is less than 0.05, showing statistically significant meaning.

It must be noted here that the above-mentioned implementation steps of the analytical methods of the present invention are not limited by the above-mentioned order. For example, the analysis method of the present invention can also first conduct step 12, then step 13, and then step 11.

By the present invention, AIF-2 can be made a biomarker for chronic kidney disease, and it is suitable as a non-invasive biomarker in early deterioration. Chronic kidney disease is divided into five phases according to the severity of the disease. The present invention shows that the AIF-2 protein can increase significantly in the IIIa phase, which reflects not only the occurrence and progression of chronic kidney disease but also the early biological index of deteriorating chronic renal failure and uremia.

Although the present invention has been explained in relation to its preferred embodiment, it is also of vital importance to acknowledge that many other possible modifications and variations can be made without departing from the spirit and scope of the invention as hereinafter claimed.

What is claimed is:

1. A method for analyzing apoptosis inducing factor-2 (AIF-2), to make AIF-2 a biomarker for chronic kidney disease, comprising the following steps:
collecting a plurality of urine samples;
conducting a western blot analysis for each urine sample of the plurality of urine samples, to detect AIF-2 protein contents in the plurality of urine samples; and
conducting a statistical analysis for the AIF-2 protein content in the plurality of urine samples, to establish AIF-2 as a biomarker for chronic kidney disease.

2. The method for analyzing apoptosis inducing factor-2 (AIF-2) as claimed in claim 1, comprising the following steps:
collecting a plurality of renal tissue samples;
conducting a immunohistochemical staining analysis for each renal tissue sample of the plurality of renal tissue samples, to detect AIF-2 protein performance conditions in the plurality of renal tissue samples; and
conducting a statistical analysis for the AIF-2 protein performance conditions in the plurality of renal tissue samples and the AIF-2 protein contents in the plurality of urine samples, to establish AIF-2 as a biomarker for chronic kidney disease.

3. The method for analyzing apoptosis inducing factor-2 (AIF-2) as claimed in claim 2, comprising the following steps:
analyzing urinary proteins, renal functions and histopathological tissues for the plurality of renal tissue samples and the plurality of urine samples.

4. The method for analyzing apoptosis inducing factor-2 (AIF-2) as claimed in claim 3, wherein the plurality of renal tissue samples and the plurality of urine samples comprise a plurality of renal tissue samples and a plurality of urine samples of patients with chronic kidney disease from phase 1 to phase 5.

5. The method for analyzing apoptosis inducing factor-2 (AIF-2) as claimed in claim 4, further comprising the following steps:
placing the plurality of urine samples on ice, obtaining supernatant of each urine sample after centrifugation and respectively pouring each urine sample into a plurality of collection tubes;
placing the plurality of renal tissue samples in liquid nitrogen; and
marking sources, names, ages, diagnoses and relevant clinical information of the plurality of urine samples and the plurality of renal tissue samples.

6. The method for analyzing apoptosis inducing factor-2 (AIF-2) as claimed in claim 2, wherein the step of conducting the immunohistochemical staining analysis further comprises:
melting wax for each renal tissue sample, slicing and conducting lost-wax, then washing by TBST;
stopping the activity of Endogenous Peroxidase;
adding 2% BSA Blocking and holding at room temperature for 30 minutes;
adding a tested primary antibody;
adding Protein G-HRP for reaction;
adding AEC and Substrate-Chromogen (DAKO) for reaction; and
soaking each renal tissue sample in a purple solution for background staining, cleaning each renal tissue sample with lean water and mounting after air drying to be observed under a microscope.

7. The method for analyzing apoptosis inducing factor-2 (AIF-2) as claimed in claim 3, wherein conducting the western blot analysis further comprises:
obtaining a supernatant after centrifuging each urine sample, adding 6X sample buffer, placing on ice and conducting a protein electrophoresis;
separating proteins on the SDS-PAGE colloid and then transfer crushing the proteins into the PVDF sigma;
placing the PVDF with the transfer crushing in 5% 1x TBST and skim milk for 2 hours and adding a first antibody at appropriate concentration dilution at 4° C. for reaction; and
adding a 2000-times diluted secondary antibody combined with HRP and shaking at room temperature for reaction for 60 minutes, adding PVDF sigma to ECL Plus and coloring with light-sensitive film.

8. The method for analyzing apoptosis inducing factor-2 (AIF-2) as claimed in claim 3, wherein the steps further comprise:
comparing the performance conditions of AIF-2 proteins of the plurality of renal tissue samples and the AIF-2 protein contents in the plurality of urine samples; and
conducting a nonparametric test or a post-mortem analysis and setting that the p value less than 0.05 for a statistically significant meaning.
